(19) United States
(12) Patent Application Publication
(10) Pub. No.: US 2009/0030065 A1
(43) Pub. Date: Jan. 29, 2009

(54) Use of Pde1c and Inhibitors Thereof

(75) Inventors: Torsten Dunkern, Stockach (DE); Armin Hatzelmann, Konstanz (DE); Friedrich Grimminger, Butzbach (DE); Ralph Schermuly, Mengerskirchen (DE)

Correspondence Address: NATH & ASSOCIATES PLLC, 112 South West Street, Alexandria, VA, 22314, US

(73) Assignee: NYCOMED GmbH, Konstanz (DE)

(21) Appl. No.: 11/921,864

(22) PCT Filed: Jun. 13, 2006
(86) PCT No.: PCT/EP2006/063138

§ 371 Date: Jan. 16, 2008

(30) Foreign Application Priority Data
Jun. 17, 2005 (EP) 05105382.5

(51) Int. Cl. A61K 31/4015; A61K 31/34; G01N 33/566; A61K 49/00

(52) U.S. Cl. 514/425; 514/471; 424/9.2; 436/501

(57) ABSTRACT

The present invention relates to the use of PDE1C as a novel target for the identification of compounds, which can be used for the treatment of pulmonary hypertension, fibrotic lung diseases or other fibrotic diseases outside the lung. The present invention further relates to the use of PDE1C inhibitors in the manufacture of pharmaceutical compositions for use in the therapy of those diseases.

### TECHNICAL FIELD

The invention relates to the use of PDE1C as a novel target for the identification of compounds that can be used for the treatment of pulmonary hypertension, fibrotic lung diseases, or other fibrotic diseases outside the lung.

The invention further relates to the use of PDE1C inhibitors in the manufacture of pharmaceutical compositions for the preventive or curative treatment of pulmonary hypertension and/or fibrotic lung diseases, or other fibrotic diseases outside the lung.

### BACKGROUND OF THE INVENTION

Pulmonary hypertension (PH) is defined by a mean pulmonary artery pressure (PAP)>25 mm Hg at rest or >30 mg Hg with exercise. According to current guidelines on diagnosis and treatment of pulmonary hypertension released by the European Society of Cardiology in 2004 (Eur Heart J 25: 2243-2278; 2004) clinical forms of PH are classified as (1) pulmonary arterial hypertension (PAH), (2) PH associated with left heart diseases, (3) PH associated with lung respiratory diseases and/or hypoxia, (4) PH due to chronic thrombotic and/or embolic disease, (5) PH of other origin (e.g. sarcoidosis). Group (1) is comprising e.g. idiopathic and familial PAH as well as PAH in the context of connective tissue disease (e.g. scleroderma, CREST), congenital systemic to pulmonary shunts, portal hypertension, HIV, intake of drugs and toxins (e.g. anorexigens). PH occurring in COPD was assigned to group (3). Muscularization of small (less than 500 μm diameter) pulmonary arterioles is widely accepted as a common pathological denominator of PAH (group 1), however it may also occur in other forms of PH such as based on COPD or thrombotic and/or thrombembolic disease. Other pathoanatomical features in PH are thickening of the intima based on migration and proliferation of (myo)fibroblasts or pulmonary smooth muscle cells and excessive generation of extracellular matrix, endothelial injury and/or proliferation and perivascular inflammatory cell infiltrates. Together, remodelling of distal pulmonary arterial vasculature results in augmented pulmonary vascular resistance, consecutive right heart failure and death. Whilst background therapy and more general measures such as oral anticoagulants, diuretics, digoxin or oxygen supply are still listed by current guidelines these remedies are not expected to interfere with causes or mechanisms of pulmonary arterial remodelling. Some patients with PAH may also benefit from Ca⁺⁺-antagonists in particular those with acute response to vasodilators. Innovative therapeutic approaches developed over the past decade considered molecular aberrations in particular enhanced endothelin-1 formation, reduced prostacyclin (PGI₂) generation and impaired eNOS activity in PAH vasculature. Endothelin-1 acting via ET_{A}-receptors is mitogenic for pulmonary arterial smooth muscle cells and triggers acute vasoconstriction. The oral ET_{A}/ET_{B}-antagonist Bosentan has recently been approved in the EU and United States for treatment of PAH after the compound demonstrated improvements in clinical endpoints such as mean PAP, PVR or 6 min walking test. However, Bosentan augmented liver enzymes and regular liver tests are mandatory. Currently selective ET_{A} antagonists such as sitaxsentan or ambrisentan are under scrutiny.

As another strategy in management of PAH replacement of deficient prostacyclin by PGI₂ analogues such as epoprostenol, treprostinil, oral beraprost or iloprost emerged. Prostacydin serves as a brake to excessive mitogenesis of vascular smooth muscle cells acting by augmenting cAMP generation. Intravenous prostacyclin (epoprostenol) significantly improved survival rates in idiopathic pulmonary hypertension as well as exercise capacity and was approved in North America and some European countries in the mid-1990s. However, owing to its short half-life epoprostenol has to be administered via continuous intravenous infusion that—whilst feasible—is uncomfortable, complicate and expensive. In addition, adverse events due to systemic effects of prostacyclin are frequent. Alternative prostacyclin analogues are treprostinil, recently approved in the United States for PAH treatment and delivered via continuous subcutaneous infusion and beraprost, the first biologically stable and orally active PGI₂ analogue, which has been approved for treatment of PAH in Japan. Therapeutic profile appeared more favourable in patients with idiopathic PAH compared to other forms of pulmonary hypertension and side effects linked to systemic vasodilation occurring following beraprost administration and local pain at the infusion site under treprostinil treatment are frequent. Administration of the prostacyclin analogue iloprost via the inhalative route was recently approved in Europe. Its beneficial effects on exercise capacity and haemodynamic parameters are to be balanced to a rather complicated dosing scheme comprising 6-12 courses of inhalation per day from appropriate devices.

Functional consequences of impaired endothelial nitric oxide formation as reported in pulmonary arterial hypertension may be overcome by selective inhibitors of phosphodiesterase-5 (PDE5) that is expressed in pulmonary artery smooth muscle cells. Consequently, the selective PDE5 inhibitor sildenafil was demonstrated to improve pulmonary haemodynamics and exercise capacity in PAH.

Most of these novel treatments primarily address smooth muscle cells function, however, in addition pulmonary vascular fibroblasts, endothelial cells but also perivascular macrophages and T-lymphocytes are considered to contribute to the development of pulmonary hypertension.

In spite of the different therapeutic approaches mentioned above the medical need to alleviate the disease burden in pulmonary hypertension is high and alternative targets to address this disease are a need.

Phosphodiesterase 1C is one of the PDE1 family members and has been shown to hydrolyze cAMP and cGMP with equal efficiency. In addition to tissue and cellular localisation this is the most prominent difference of PDE1C in comparison to PDE1A and B. Five splicing variants of PDE1C (1C1, 1C2, 1C3, 1C4, 1C5) has been identified up to now which are expressed in a tissue specific manner (Yan et al., Journal of Biological Chemistry, 271, 25699-25706, 1996). PDE1C has been shown to be induced in proliferating smooth muscle cells of the aorta (Rybalkin et al., J. Clin. Invest, 100, 2611-2621, 1997) and down-regulation of PDE1C by antisense-technology has been shown to reduce proliferation in this cells (Rybalkin et al., Circ. Res., 90, 151-157, 2002). The expression of PDE1C in smooth muscle cells of other origin has not been analyzed up to now. Within this invention we demonstrate PDE1C to be a therapeutic target for the treatment of pulmonary hypertension.

The international application WO2004/031375 describes a human PDE1C (and its use), which is said to can play a role in treating diseases, including, but not limited thereto, cancer, diabetes, neurological disorders, asthma, obesity or cardiovascular disorders.

The international application WO2004/080347 describes a human PDE1C (and its use), which is said to be associated with cardiovascular disorders, gastrointestinal and liver diseases, cancer disorders, neurological disorders, respiratory diseases and urological disorders.

The US application US2002160939 describes methods of identifying novel agents that increase glucose dependent insulin secretion in pancreatic islet cells as well as methods of treating diabetes using the agents which have an inhibitory effect on the activity of pancreatic islet cell PDE enzyme, namely PDE1C.

### DESCRIPTION OF THE INVENTION

Unanticipatedly and unexpectedly R has now been found, that treatment of pulmonary hypertension can be achieved by the use of inhibitors of phosphodiesterase 1C (PDE1C).

Yet unanticipatedly and unexpectedly it has now been found, that treatment of fibrotic lung diseases can be achieved by the use of inhibitors of phosphodiesterase 1C (PDE1C).

Furthermore, for the first time, the present invention provides evidence and data for the efficiency of inhibitors of PDE1C for the treatment of the diseases mentioned herein.

Yet furthermore, for the first time, the present invention provides evidence and data for a mechanistical involvement of PDE1C in the diseases mentioned herein.

Thus e.g., it is shown herein, that PDE1C inhibitors block proliferation of cells involved in remodelling process observed in pulmonary hypertension and also in-vivo data are provided.

Consequently, the present invention discloses for the first time the usability of selective PDE1C inhibitors for the therapy of any one of the diseases mentioned herein.

Moreover, for the first time, the present invention discloses representatively certain structures of selective PDE1C inhibitors.

Further on, the present invention discloses the suitability of PDE1C for identifying a compound which can be used for the treatment of pulmonary hypertension, lung diseases associated with an increased proliferation of pulmonary fibroblasts, or non-lung diseases associated with an increased proliferation of fibroblasts; such as e.g. any of those diseases mentioned herein, particularly pulmonary hypertension or fibrotic lung diseases.

According to this invention, a substance is considered to be a PDE1C inhibitor as used herein if it has an IC₅₀ against PDE1C of less than or about 1 μM, in another embodiment, less than or about 0.1 μM, in yet another embodiment, less than or about 0.01 μM, in still yet another embodiment, less than or about 1 nM.

In an embodiment of this invention, the meaning of a PDE1C inhibitor as used herein refers to a PDE inhibitor, which inhibits preferentially the type 1C phosphodiesterase (PDE1C) when compared to other known types of phosphodiesterase, e.g. any enzyme from the PDE families. According to this invention, a PDE inhibitor preferentially inhibiting PDE1C refers to a compound having a lower IC₅₀ for the type 1C phosphodiesterase compared to IC₅₀ for inhibition of other known type of phosphodiesterase, such as, for example, wherein the IC₅₀ for PDE1C inhibition is about factor 10 lower than the IC₅₀ for inhibition of other known types of phosphodiesterase, and therefore is more potent to inhibit PDE1C.

In a preferred embodiment of this invention, the meaning of a PDE1C inhibitor as used herein refers to a selective PDE1C inhibitor.

In one detail of this invention, the meaning of a selective PDE1C inhibitor as used herein refers to a compound, which inhibits the type 1C phosphodiesterase (PDE1C) at least ten times more potent than other PDE family members.

In a further detail of this invention, the meaning of a selective PDE1C inhibitor as used herein refers to a compound, which inhibits the type 1C phosphodiesterase (PDE1C) at least ten times more potent than any enzyme of the PDE 2 to 11 families.

In yet a further detail of this invention, the meaning of a selective PDE1C inhibitor as used herein refers to a compound, which inhibits the type 1C phosphodiesterase (PDE1C) at least ten times more potent than any other enzyme of the PDE 1 to 11 families.

PDE1C inhibitors as used herein can be identified as it is known to the person skilled in the art or as described in the present invention, e.g. comprising using the mentioned methods, processes and/or assays.

In another embodiment of this invention, the meaning of a PDE1C inhibitor as used herein refers to a compound that only or essentially only inhibits the PDE1C enzyme, not a compound which inhibits to a degree of exhibiting a therapeutic effect also other members of the PDE enzyme family.

Methods to determine the activity and selectivity of a phosphodiesterase inhibitor are known to the person skilled in the art. In this connection it may be mentioned, for example, the methods described by Thompson et al. (Adv Cycl Nucl Res 10: 69-92, 1979), Giembycz et al. (Br J Pharmacol 118: 1945-1958, 1996) and the phosphodiesterase scintillation proximity assay of Amersham Pharmacia Biotech.

Within this invention data are provided that human pulmonary arterial smooth muscle cells and human pulmonary fibroblasts express cAMP—as well as cGMP-calmodulin-stimulated phosphodiesterase activity due to the expression of PDE1C. Furthermore this invention demonstrates surprisingly a strong up-regulation of the expression of PDE1C mRNA and protein in the lung issue of patients with idiopathic pulmonary hypertension in comparison to lung tissue of healthy donors. In addition the same up-regulation of PDE1C mRNA and protein is shown in lung issue of hypoxic kept mice, which are developing pulmonary hypertension and to some degree reflect the pathophysiological conditions observed in patients with pulmonary hypertension. Enhanced PDE1C expression in patients and within the lung of the animal model is shown to be localized in pulmonary smooth muscle cells of the medial wall of small pulmonary vessels undergoing strong remodeling processes, which ultimately lead to enhanced vascular resistance and thus pulmonary hypertension. Furthermore enhanced expression of PDE1C correlates with the extent of pulmonary arterial pressure. In addition PDE1C inhibitors shown in this invention inhibit proliferation of PDE1C expressing human pulmonary fibroblasts and human pulmonary arterial smooth muscle cells as shown below.

Based on this data and the known function of PDE1C in the control of proliferation selective inhibitors of PDE1C can be used to inhibit proliferation mediated remodeling processes of the lung vasculature (and neighboured tissues) of patients with primary and secondary pulmonary hypertension.

The expression “pulmonary hypertension” as used herein comprises different forms of pulmonary hypertension. Non-limiting examples, which may be mentioned in this connection are idiopathic pulmonary arterial hypertension; familial pulmonary arterial hypertension; pulmonary arterial hypertension associated with collagen vascular disease, congenital systemic-to-pulmonary shunts, portal hypertension, HIV infection, drugs or toxins; pulmonary hypertension associated with thyroid disorders, glycogen storage disease, Gaucher disease, hereditary hemorrhagic telangiectasia, hemoglobinopathies, myeloproliferative disorders or splenectomy; pulmonary arterial hypertension associated with pulmonary capillary hemangiomatosis; persistent pulmonary hypertension of the newborn; pulmonary hypertension associated with chronic obstructive pulmonary disease, interstitial lung disease, hypoxia driven alveolar hypoventlation disorders, hypoxia driven sleep-disordered breathing or chronic exposure to high altitude; pulmonary hypertension associated with development abnormalities; and pulmonary hypertension due to thromboembolic obstruction of distal pulmonary arteries.

Based on the unexpected expression of PDE1C in human pulmonary fibroblasts PDE1C inhibitors can be used for the treatment of lung diseases associated with an increased proliferation of human pulmonary fibroblasts, such as e.g. fibrotic lung diseases.

In the context of this finding, PDE1C inhibitors might be also used for the treatment of other diseases associated with an increased proliferation of human fibroblasts in general, e.g. fibrotic diseases outside the lung, such as, for example, (diabetic) neprophropathy, glomerulonephritis, myocardial fibrosis, cardiac valve disease, liver fibrosis, pancreatitis, Dupuytren's disease (palmar fascia fibrosis), peritoneal fibrosis (e.g. based on long-term peritoneal dialysis), Peyronie's disease or collagenous colitis.

Moreover, as a further consequence of the data disclosed herein, the present invention provides a novel use of PDE1C for identifying a compound which can be used for the treatment of pulmonary hypertension and/or fibrotic lung diseases, or fibrotic diseases outside the lung, such as e.g. those described above.

The present invention also provides a process for identifying and obtaining a compound for therapy of pulmonary hypertension and/or fibrotic lung diseases, said process comprising measuring the PDE1C inhibitory activity and/or selectivity of a compound suspected to be a PDE1C inhibitor, and a compound identified by said process. Advantageously, said compound may be a selective PDE1C inhibitor.

Said process may also comprise administering a compound suspected to be a PDE1C inhibitor to an animal, preferably a non-human animal, in which pulmonary hypertension is induced, and measuring the extent of pulmonary hypertension as compared to control-treated animals. Advantageously, said compound may be a selective PDE1C inhibitor.

Corresponding procedures are well known in the art or are described by way of example in the following examples.

Optionally comprised in said process, in a first option, the compounds identified as hereinbefore described may be formulated with a pharmaceutically acceptable carrier or diluent.

Yet optionally comprised in said process, in an alternative option, the compounds identified as hereinbefore described may be modified to achieve (i) modified site of action, spectrum of activity, and/or (ii) improved potency, and/or (iii) decreased toxicity (improved therapeutic index), and/or (iv) decreased side effects, and/or (v) modified onset of action, duration of effect, and/or (vi) modified kinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetates, or (xiv) N-acetylation to amides, phenylcarbamates, or (xv) synthesis of Mannich bases, imines, or (xvi) transformation of ketones or aldehydes to Schiff s bases, oximes, acetates, ketales, enolesters, oxazolidines, thiozolidines or combinations thereof; and, optionally, formulating the product of said modification with a pharmaceutically acceptable carrier or diluent.

A compound suspected to be a PDE1C inhibitor as used herein may be, for example, without being limited thereto, a selective PDE1 inhibitor known from the art, such as e.g. any compound which inhibits PDE1 at least ten times more potent than other PDE family members.

Further on, a compound suspected to be a PDE1C inhibitor as used herein may be, for example, without being limited thereto, any compound which is developed as a PDE inhibitor, such as e.g. a compound for which PDE1 inhibitory activity is found.

Yet further on, a compound suspected to be a PDE1C inhibitor as used herein may be, for example, without being limited thereto, any compound whose PDE inhibitory profile is to be assayed.

Still yet further on, a compound suspected to be a PDE1C inhibitor as used herein may be, for example, without being limited thereto, any compound which is contained in a commercially available compound library.

The present invention also pertains to a compound identified by any of the processes herein described.

As a medicament (also referred to as pharmaceutical preparation, formulation or composition herein), the PDE1C inhibitor is either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions. The pharmaceutical preparation of the invention typically comprises a total amount of active compound in the range from 0.05 to 99% w (percent by weight), more preferably in the range from 0.10 to 70% w, even more preferably in the range from 0.10 to 50% w, all percentages by weight being based on total preparation. By the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, vehicles, excipients, diluents, carriers or adjuvants which are suitable for the desired pharmaceutical formulations on account of his/her expert knowledge. In addition to solvents, gel formers, ointment bases and other active compound excipients, for example antioxidants, dispersants, emulsifiers, preservatives, solubilizers, colorants, complexing agents, flavours, buffering agents, viscosity-regulating agents, surfactants, binders, lubricants, stabilizers or permeation promoters, can be used.

The PDE1C inhibitor may be administered to a patient in need of treatment in any of the generally accepted modes of administration available in the art. Illustrative examples of suitable modes of administration include oral, intravenous, nasal, parenteral, transdermal and rectal delivery as well as administration by inhalation. Preferred modes of administration are oral and inhalation.

The amount of a PDE1C inhibitor which is required to achieve a therapeutic effect will, of course, vary with the particular compound, the route of administration, the subject under treatment, and the particular disorder or disease being treated. In general, the daily dosage will generally range from about 0.001 to about 100 mg/kg body weight. As an example, a PDE1C inhibitor may be administered orally to adult humans at a dose from about 0.1 to about 1000 mg daily, in single or divided (i.e. multiple) portions.

Thus, a first aspect of the present invention is the use of a PDE1C inhibitor for the production of a pharmaceutical composition for the preventive or curative treatment of pulmonary hypertension.

In a second aspect the present invention relates to a method for the preventive or curative treatment of pulmonary hypertension in a patient comprising administering to said patient an effective amount of a PDE1C inhibitor.

In a third aspect of the present invention relates to the use of a PDE1C inhibitor for the production of a pharmaceutical composition for the treatment of lung diseases associated with an increased proliferation of human pulmonary fibroblasts, such as e.g. fibrotic lung diseases.

In a fourth aspect the present invention relates to a method for the treatment of lung diseases associated with an increased proliferation of human pulmonary fibroblasts, such as e.g. fibrotic lung diseases, in a patient comprising administering to said patient an effective amount of a PDE1C inhibitor.

In a fifth aspect of the present invention relates to the use of a PDE1C inhibitor for the production of a pharmaceutical composition for the treatment of non-lung diseases associated with an increased proliferation of human fibroblasts, e.g. fibrotic diseases outside the lung, such as, for example, (diabetic) neprophropathy, glomerulonephritis, myocardial fibrosis, cardiac valve disease, liver fibrosis, pancreatitis, Dupuytren's disease (palmar fascia fibrosis), peritoneal fibrosis (e.g. based on long-term peritoneal dialysis), Peyronie's disease or collagenous colitis.

In a sixth aspect the present invention relates to a method for the treatment of non-lung diseases associated with an increased proliferation of human fibroblasts, e.g. fibrotic diseases outside the lung, such as, for example, (diabetic) neprophropathy, glomerulonephritis, myocardial fibrosis, cardiac valve disease, liver fibrosis, pancreatitis, Dupuytren's disease (palmar fascia fibrosis), peritoneal fibrosis (e.g. based on long-term peritoneal dialysis), Peyronie's disease or collagenous colitis, in a patient comprising administering to said patient an effective amount of a PDE1C inhibitor.

In an eighth aspect the present invention relates to the use of PDE1C for identifying a compound which can be used for the treatment of pulmonary hypertension, fibrotic lung diseases, or fibrotic diseases outside the lung.

In a ninth aspect the present invention relates to a method for identifying a compound useful for the treatment of pulmonary hypertension and/or fibrotic lung diseases, which method comprises determining for said compound its PDE1C inhibitory activity and/or selectivity.

The term “effective amount” refers to a therapeutically effective amount of a PDE1C inhibitor.

“Patient” includes both human and other mammals.

The present invention also provides the compounds, processes, uses and compositions substantially as hereinbefore described, especially with reference to the examples.

### Pharmacology

Characterisation of PDE1C Expression in the Lung of Healthy Humans, Patients with Idiopathic Pulmonary Hypertension and Hypoxic/Normoxic Mice.

### Objective

The objective of the pharmacological investigation was to characterize the expression and localization of PDE1C in the lung of patients with idiopathic pulmonary hypertension and compare them with that of healthy humans. PDE1C expression was correlated with the degree of pulmonary hypertension in the patient group. Similar analysis were performed on hypoxic/normoxic mice used as an animal model for pulmonary hypertension.

### Patient Characteristics

Human lung tissue was obtained from five healthy lung donors and five PAH patients (all idiopathic PAH) which underwent lung transplantation. Patient lung tissue was snap frozen directly after explanation for mRNA and protein extraction or directly transferred into 4% buffered paraformaldehyde, fixed for 24 h at 4° C. and embedded in paraffin. Mean pulmonary arterial pressure of the IPAH patients under investigation was 68.4±8.5 mmHg. Tissue donation was regulated by the Justus-Liebig University Ethical Committee and national law.

### Cell Culture

Human pulmonary smooth muscle cells were obtained from Promocell GmbH (Hdbg. Germany) and cultured for up to three passages in human smooth muscle cell medium II (Promocell GmbH, Hdbg., Germany). Human lung fibroblasts were obtained from Cambrex Bioscience and cultured in fibroblast growth medium (Cambrex Bioscience). A549 cells were culture in Dulbecco's modified eagle medium containing 10% fetal calf serum.

### Animals

All animal experiments were performed using adult male mice (8-week-old BALB/c) according to the institutional guidelines that comply with national and international regulations.

### Exposure to Chronic Hypoxia

Mice were exposed to chronic hypoxia (10% O₂) in a ventilated chamber, as described previously¹⁶. The level of hypoxia was held constant by an auto regulatory control unit (model 4010, O₂ controller, Labotect; Göttingen, Germany) supplying either nitrogen or oxygen. Excess humidity in the recirculating system was prevented by condensation in a cooling system. CO₂ was continuously removed by soda lime. Cages were opened once a day for cleaning as well as for food and water supply. The chamber temperature was maintained at 22-24° C. Normoxic mice were kept in identical chambers under normoxic condition.

### Hemodynamic Measurements

Mice were anaesthetized with ketamine (6 mg/100 g, intraperitoneally) and xylazine (1 mg/100 g, intraperitoneally). The trachea was cannulated, and the lungs were ventilated with room air at a tidal volume of 0.2 ml and a rate of 120 breaths per minute. Systemic arterial pressure was determined by catheterization of the carotid artery. For measurement of right ventricular systolic pressure (RVSP) a PE-80 tube was inserted into the right ventricle via the right vena jugularis.

### Pharmacologic Treatments

To investigate the effects of a PDE1C inhibitor on acute hypoxic vasoconstriction, four groups of mice (six in each group) are studied in isolated lung experiments. Two groups are normoxic animals in which the effect of increasing doses of the test compound or placebo on acute hypoxic pulmonary vasoconstriction is investigated. Therefore, repetitive hypoxic challenges are performed and the test compound or placebo is applied in the normoxic periods. The other two groups consisted of chronically hypoxic mice (21 days at 10% O₂) in which identical experiments with the test compound or placebo are performed.

The chronic effects of PDE1C inhibition are assessed in mice exposed to hypoxia for 35 days. Briefly, 20 animals are kept in hypoxic conditions to develop pulmonary hypertension. After 21 days, animals are randomized to receive either the test compound or placebo via continuous infusion by implantation of osmotic minipumps. Animals are anaesthetized with ketamine/xylazine and a catheter inserted into the jugular vein. The animals receive either 20 μg test compound/kg/min or placebo for 14 days.

### Assessment of Right Heart Hypertrophy and Vascular Remodeling

Hemodynamics of mice exposed to hypoxia or room air for 3 or 5 weeks were recorded as described above. After recording systemic arterial and right ventricular pressure, the animals were exsanguinated and the lungs and heart were isolated. The RV was dissected from the left ventricle+septum (LV+S) and these dissected samples were weighed to obtain the right to left ventricle plus septum ratio (RV/LV+S).

The lungs were perfused with a solution of 10% phosphate buffered formalin (pH 7.4). At the same time 10% phosphate buffered formalin (pH 7.4) was administered into the lungs via the tracheal tube at a pressure of 20 cm H₂O and processed for light microscopy. The degree of muscularization of small peripheral pulmonary arteries was assessed by double-staining the 3 μm sections with an anti-smooth muscle actin antibody (dilution 1:900, clone 1A4, Sigma, Saint Louis, Mo.) and anti-human von Willebrand factor antibody (vWF, dilution 1:900, Dako, Hamburg, Germany) modified from a protocol described elsewhere¹⁹. A polyclonal antibody against human PDE1C (FabGennix, Shreveprot, USA) raised in rabbits was used for PDE1C staining. Dewaxed and rehydrated sections were subjected to proteolytic antigen retrieval with 0.1% trypsin in 0.1% calcium chloride (pH 7.6) at 37° C. for 8 minutes and immunostained with the avidin-biotin-peroxidase complex (ABC Elite, Vector Laboratories, Burlingame, USA) method, with 3,3-diaminobenzidine as substrate. Sections were counterstained with hematoxylin and examined by light microscopy using a computerized morphometric system (Qwin, Leica, and Wetzlar, Germany). At 40× magnification 50-60 intraacinar vessels accompanying either alveolar ducts or alveoli were analyzed by an observer blinded to treatment in each mouse. As described, each vessel was categorized as nonmuscularized, partially muscularized or fully muscularized²⁰. The percentage of pulmonary vessels in each muscularization category was determined by dividing the number of vessels in that category by the total number counted in the same experimental group.

### Western Blot

Frozen lung tissue was homogenized with a tissue homogenizer in a Tris lysis buffer containing 50 mM Tris-HCl pH 7.6, 10 mM CaCl₂, 150 mM NaCl, 60 mM NaN₃ and 0.1% w/v Triton X-100 with a protease cocktail inhibitor (Roche, Mannheim, Germany). The homogenized sample was centrifuged at 10,000 g for 30 min and the supernatant was collected and the protein content was estimated by Bradford's dye reagent method. Briefly equal amount of protein was loaded on a 12% SDS PAGE after boiling the sample at 95° C. for 5 min in SDS sample buffer containing β-mercaptoethanol. The gel was then transferred on to a nitrocellulose membrane and the membrane was incubated with PDE1C (FabGennix, Shreveprot, USA) and smooth muscle actin antibody (Sigma, Munich, Germany) respectively. The membrane was developed using ECL chemiluminescene kit (Amersham, Freiburg, Germany).

### Reverse-Transcription Polymerase Chain Reaction

Total RNA was isolated from frozen lung tissues by TRizol method (Invitrogen GmbH, Karlsruhe Germany) and the quantity of RNA was measured using nanodrop (NanoDrop ND-1000, Wilmington, USA). Reverse transcription polymerase chain reaction (RT-PCR) was performed using oligo dt primer to generate first strand cDNA. Semi quantitative PCR was performed using the following oligonucleotide primers to check the mRNA expression of PDE1C gene. For the expression of human PDE1C a primer pair with sense sequence HPDE1CF-5′-AAACTGGTGGGACAGGACAG-3′ and an antisense sequence of HPDE1CR-5′-ACTTTTGTTTGCCCGTGTTC-3′ were used. Similarly for the mRNA expression of PDE1C in mouse a primer pair with the following sequence were used forward MPDE1C-5′-TTGACGAAAGCTCCCAGACT-3′ and reverse MPDE1C-5′-TTCAAGTCACCGTTCTGCTG-3′. Beta actin was used as a house keeping gene for both the organism with a common primer set of forward β-ACTINF-5′-CGAGCGGGAAATCGTGCGTGACATTAAGGAGA-3′ and reverse β-ACTINR-5′-CGTCATACTCCTGCTTGCTGATCCACATCTGC-3′. The PCR was carried out under the following conditions. An initial denaturation at 94° C. for 1 min. 30 sec, annealing at 58° C. for 1 min, polymerisation at 72° C. for 1 min 20 sec for 32 cycles and a final extension at 72° C. for 2 min. Human PDE1C primer yielded an amplicon size of 377 bp and mice PDE1C primer amplified 450 bp, whereas Beta actin gave a product size of 475 bp.

### Measurements of Phosphodiesterase Isoenzyme Activities and Preparation of Cellular Extracts

Cells (1-3×10⁶) were washed twice in phosphate buffered saline (4° C.) and resuspended in 1 ml homogenization buffer (137 mM NaCl, 2.7 mM KCl, 8.1 mM Na₂HPO4, 1.5 mM KH₂PO₄, 10 mM HEPES, 1 mM EGTA, 1 mM MgCl₂, 1 mM-mercaptoethanol, 5 mM pepstatin A, 10 mM leupeptin, 50 mM phenylmethylsulfonyl fluoride, 10 mM soybean trypsin inhibitor, 2 mM benzamidine, pH 8.2). Cells were disrupted by sonication (Branson sonifier, 3×15 s) and lysates were immediately used for phosphodiesterase (PDE) activity measurements. PDE activities were assessed in cellular lysates as described (Thompson & Appleman, 1979) with some modifications (Bauer & Schwabe, 1980). The assay mixture (final volume 200 ml) contained (mM): Tris HCl 30; pH 7.4, MgCl₂ 5, 0.5 μM either cyclic AMP or cyclic GMP as substrate including [³H]cAMP or [³H]cGMP (about 30 000 c.p.m. per well), 100 mM EGTA, PDE isoenzyme-specific activators and inhibitors as described below and cellular lysates. Incubations were performed for 60 min at 37° C. and reactions were terminated by adding 50 ml 0.2 M HCl per well. Assays were left on ice for 10 min and then 25 mg 5′-nucleotidase (Crotalus atrox) was added. Following an incubation for 10 min at 37° C. assay mixtures were loaded onto QAE-Sephadex A25 columns (1 ml bed volume). Columns were eluted with 2 ml 30 mM ammonium formiate (pH 6.0) and radioactivity in the eluate was counted. Results were corrected for blank values (measured in the presence of denatured protein) that were below 2% of total radioactivity. cyclic AMP degradation did not exceed 25% of the amount of substrate added. The final DMSO concentration was 0.3% (v/v) in all assays. Selective inhibitors and activators of PDE isoenzymes were used to determine activities of PDE families as described previously (Rabe et al., 1993) with modifications. Briefly, PDE4 was calculated as the difference of PDE activities at 0.5 μM cyclic AMP in the presence and absence of 1 μM Piclamilast. The difference between Piclamilast-inhibited cyclic AMP hydrolysis in the presence and absence of 10 μM Motapizone was defined as PDE3. The fraction of cyclic GMP (0.5 μM) hydrolysis in the presence of 10 μM Motapizone that was inhibited by 100 nM Sildenafil reflected PDE5. At the concentrations used in the assay Piclamilast (1 μM), Motapizone (10 μM) and Sildenafil (100 nM) completely blocked PDE4, PDE3 and PDE5 activities without interfering with activities from other PDE families. PDE1 was defined as the increment of cyclic AMP hydrolysis (in the presence of 1 μM Piclamilast and 10 μM Motapizone) or cyclic GMP hydrolysis induced by 1 mM Ca²⁺ and 100 nM calmodulin. The increase of cyclic AMP (0.5 μM) degrading activity in the presence of 1 μM Piclamilast and 10 μM Motapizone induced by 5 μM cyclic GMP represented PDE2. The PDE2 inhibitor PDP (100 nM) completely inhibited this cyclic GMP-induced activity increment further verifying this activity as PDE2.

### Proliferation Measurement

Proliferation was measured by means of ³H-thymidine incorporation. 2.4×10⁴ human pulmonary arterial smooth muscle cells or human pulmonary fibroblasts were seeded per well in 24 well-plates. One day after seeding PDE1C-inhibitors (compound A and compound B) were added. Depending on the experiment one day or three days after adding the compounds ³H-thymidine was added to each well and cells were further incubated for at least 10 hours. After discarding the medium supernatant, cells were washed twice with 1 ml of PBS. Thereafter 10% TCA was added for 30 min. This was followed by adding 0.5 ml 0.2 M NaOH for at least 15 hours at 4° C. Thereafter samples were transferred to scintillation vials, 5 ml scintillation fluid was added and vials were counted on a Multi Purpose Scintillation Counter LS6500 (Beckman Coulter).

Proliferation assays with A549 cells were performed in a different way in 96 well plates. Briefly 5,000 cells per well were seeded in 100 μl. One day after the PDE1C inhibitors (compound A and compound B) were added for 8 hours which was followed by adding ³H-thymidine for 2 hours. Thereafter the supernatant was discarded, cells were trypsinized and sucked on 96 well-filter plate by using a filtermate harvester (Packard Bioscience). Thereafter 30 μl of scintillation fluid was added to each well of the filter plate, the plate was covered by attaching a film on the top of the plate and plate was measured on a Top Count NXT™ (Packard Bioscience).

Measurement of the Inhibition of Phosphodiesterase Activity Phosphodiesterase activity is measured in a modified SPA (scintillation proximity assay) test, supplied by Amersham Biosciences (see procedural instructions “phosphodiesterase [3H]cAMP SPA enzyme assay, code TRKQ 7090”), carried out in 96-well microtitre plates (MTP's): The test volume is 100 μl and contains 20 mM Tris buffer (pH 7.4), 0.1 mg of BSA (bovine serum albumin)/ml, 5 mM Mg²⁺, 0.5 μM cGMP or cAMP (including about 50,000 cpm of [3H]cGMP or [3H]cAMP as a tracer; whether to use cAMP or cGMP depends on the substrate-specificity of the phosphodiesterase measured), 1 μl of the respective substance dilution in DMSO and sufficient recombinant PDE to ensure that 10-20% of the cGMP or cAMP is converted under the said experimental conditions. The final concentration of DMSO in the assay (1% v/v) does not substantially affect the activity of the PDE investigated. After a preincubation of 5 min at 37° C., the reaction is started by adding the substrate (cGMP) and the assay is incubated for a further 15 min; after that, it is stopped by adding SPA beads (50 μl). In accordance with the manufacturer's instructions, the SPA beads had previously been resuspended in water, but were then diluted 1:3 (v/v) in water; the diluted solution also contains 3 mM IBMX to ensure a complete PDE activity stop. After the beads have been sedimented (>30 min), the MTP's are analyzed in commercially available luminescence detection devices. The corresponding IC₅₀ values of the compounds for the inhibition of PDE activity are determined from the concentration-effect curves by means of non-linear regression.

### PDE1C Inhibitors Inhibit Proliferation of PDE1C Expressing Lung Cells.

Compounds are identified that inhibit the activity of PDE1C. The compounds include the compounds A and B having the formulae as shown below.

Compound A and B are analyzed for inhibition of PDE family members as described. Both compounds turn out to inhibit human recombinant PDE1C1 with an IC₅₀ value in the nanomolar range and to be selective versus other PDE family members tested (see Tab. 1).

TABLE 1
Structures and IC₅₀ values of compound A and B on human
recombinant phosphodiesterase enzymes.

| | Compound A | Compound B |
|---|---|---|
| PDE | IC₅₀ (nM) | IC₅₀ (nM) |
| 1C1 | 83 | 100 |
| 2A3 | >100000 | 13000 |
| 3A1 | >100000 | >100000 |
| 4B2 | >100000 | 9300 |
| 5A1 | >100000 | 16000 |
| 10A | >100000 | 77000 |
| 11A4 | >100000 | 22000 |

Compound A:

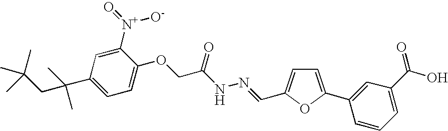

Compound B:

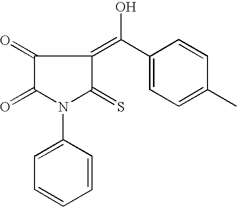

4-[Hydroxy(4-methylphenyl)methylidene]-1-phenyl-5-thioxopyrrolidine-2,
3-dione

1. (canceled)

2. A method for the preventive or curative treatment of pulmonary hypertension in a patient comprising administering to said patient an effective amount of a PDE1C inhibitor.

3. The method according to claim 1, in which pulmonary hypertension is selected from the group consisting of idiopathic pulmonary arterial hypertension; familial pulmonary arterial hypertension; pulmonary arterial hypertension associated with collagen vascular disease, congenital systemic-to-pulmonary shunts, portal hypertension, HIV infection, drugs or toxins; pulmonary hypertension associated with thyroid disorders, glycogen storage disease, Gaucher disease, hereditary hemorrhagic telangiectasia, hemoglobinopathies, myeloproliferative disorders or splenectomy; pulmonary arterial hypertension associated with pulmonary capillary hemangiomatosis; persistent pulmonary hypertension of the newborn; pulmonary hypertension associated with chronic obstructive pulmonary disease, interstitial lung disease, hypoxia driven alveolar hypoventilation disorders, hypoxia driven sleep-disordered breathing or chronic exposure to high altitude; pulmonary hypertension associated with development abnormalities; and pulmonary hypertension due to thromboembolic obstruction of distal pulmonary arteries.

4. A method for the treatment of lung diseases associated with an increased proliferation of pulmonary fibroblasts in a patient comprising administering to said patient an effective amount of a PDE1C inhibitor.

5. A method for the treatment of non-lung diseases associated with an increased proliferation of fibroblasts in a patient comprising administering to said patient an effective amount of a PDE1C inhibitor.

6. The method according to claim 2 wherein the PDE1C inhibitor is a selective PDE1C inhibitor which inhibits the type 1C phosphodiesterase (PDE1C) at least ten times more potent than other PDE family members.

7. (canceled)

8. (canceled)

9. A process for identifying and obtaining a compound useful for the treatment of pulmonary hypertension and/or fibrotic lung diseases comprising
measuring the PDE1C inhibitory activity and/or selectivity of a compound suspected to be a PDE1C inhibitor; and/or
administering a compound suspected to be a PDE1C inhibitor to a non-human animal in which pulmonary hypertension is induced, and measuring the extent of pulmonary hypertension as compared to control-treated animals.

10. A composition made by combining a compound identified by the process according to claim 9 and a pharmaceutically acceptable auxiliary, diluent or carrier.

11. A method for the treatment of pulmonary hypertension and/or fibrotic lung diseases in a patient administering a compound identified by the process according to claim 9 to said patient.
